(19) United States
(12) Patent Application Publication
(10) Pub. No.: US 2024/0361298 A1
(43) Pub. Date: Oct. 31, 2024

(54) VEHICLE

(71) Applicant: SUBARU CORPORATION, Tokyo (JP)

(72) Inventor: Satoru IWATA, Tokyo (JP)

(73) Assignee: SUBARU CORPORATION, Tokyo (JP)

(21) Appl. No.: 18/637,246

(22) Filed: Apr. 16, 2024

(30) Foreign Application Priority Data
Apr. 25, 2023 (JP) 2023-071368

(51) Int. Cl. G01N 33/497; B60H 1/00; B60H 1/26

(52) U.S. Cl. CPC G01N 33/4972; B60H 1/00878; B60H 1/265

(57) ABSTRACT

A vehicle includes an alcohol sensor, a blower, and an air duct. The alcohol sensor is configured to detect an alcohol component included in exhaled breath of an occupant of the vehicle. The blower is configured to blow air, and adjusts a temperature of the air to be blown out from the blower. The air duct is configured to flow the air blown out from the blower toward the alcohol sensor.

### CROSS-REFERENCE TO RELATED APPLICATIONS

The present application claims priority from Japanese Patent Application No. 2023-071368 filed on Apr. 25, 2023, the entire contents of which are hereby incorporated by reference.

### BACKGROUND

The disclosure relates to a vehicle configured to detect an alcohol component included in exhaled breath of an occupant of the vehicle.

Some vehicles have an alcohol sensor that determines whether an occupant has drunk alcohol. Some alcohol sensors detect an alcohol component included in exhaled breath of the occupant. For example, Japanese Unexamined Patent Application Publication No. 2005-118177 discloses a technique in which a heater is provided, and a temperature of the alcohol sensor is so adjusted that the alcohol sensor operates within a predetermined operation temperature range.

### SUMMARY

An aspect of the disclosure provides a vehicle that includes an alcohol sensor, a blower, and an air duct. The alcohol sensor is configured to detect an alcohol component included in exhaled breath of an occupant of the vehicle. The blower is configured to blow air, and adjusts a temperature of the air to be blown out from the blower. The air duct is configured to flow the air blown out from the blower toward the alcohol sensor.

### BRIEF DESCRIPTION OF THE DRAWINGS

The accompanying drawings are included to provide a further understanding of the disclosure, and are incorporated in and constitute a part of this specification. The drawings illustrate embodiments and, together with the specification, serve to explain the principles of the disclosure.

FIG. 1 is an explanatory diagram illustrating a configuration example of a vehicle according to one example embodiment of the disclosure.

FIG. 2 is a block diagram illustrating a configuration example of the vehicle illustrated in FIG. 1.

FIG. 3 is an explanatory diagram illustrating a configuration example of a vehicle compartment of the vehicle illustrated in FIG. 1.

FIG. 4 is a cross-sectional diagram illustrating a configuration example of a door illustrated in FIG. 3.

FIG. 5 is an explanatory diagram illustrating a configuration example of the vehicle compartment of the vehicle according to a modification example.

FIG. 6 is an explanatory diagram illustrating a configuration example of the vehicle compartment of the vehicle according to a modification example.

FIG. 7 is a block diagram illustrating a configuration example of a vehicle according to a modification example.

FIG. 8 is an explanatory diagram illustrating a configuration example of a vehicle compartment of the vehicle illustrated in FIG. 7.

FIG. 9 is a cross-sectional diagram illustrating a configuration example of a door according to a modification example.

### DETAILED DESCRIPTION

What is desired for a vehicle is that an occupant can easily use an alcohol sensor, and that the alcohol sensor is more easily usable.

It is desirable to provide a vehicle that makes it possible for an occupant to easily use an alcohol sensor.

In the following, some example embodiments of the disclosure are described in detail with reference to the accompanying drawings. Note that the following description is directed to illustrative examples of the disclosure and not to be construed as limiting to the disclosure. Factors including, without limitation, numerical values, shapes, materials, components, positions of the components, and how the components are coupled to each other are illustrative only and not to be construed as limiting to the disclosure. Further, elements in the following example embodiments which are not recited in a most-generic independent claim of the disclosure are optional and may be provided on an as-needed basis. The drawings are schematic and are not intended to be drawn to scale. Throughout the present specification and the drawings, elements having substantially the same function and configuration are denoted with the same reference numerals to avoid any redundant description. In addition, elements that are not directly related to any embodiment of the disclosure are unillustrated in the drawings.

FIG. 1 illustrates a configuration example of a vehicle 1 according to an example embodiment. The vehicle 1 includes an alcohol sensor 10. The alcohol sensor 10 is configured to detect an alcohol component included in exhaled breath of a person such as a driver who drives the vehicle 1 or an occupant. The alcohol sensor 10 may detect the alcohol component included in the exhaled breath of the driver, based on, for example, blowing of a breath of the driver toward a later-described inlet 10A of the alcohol sensor 10.

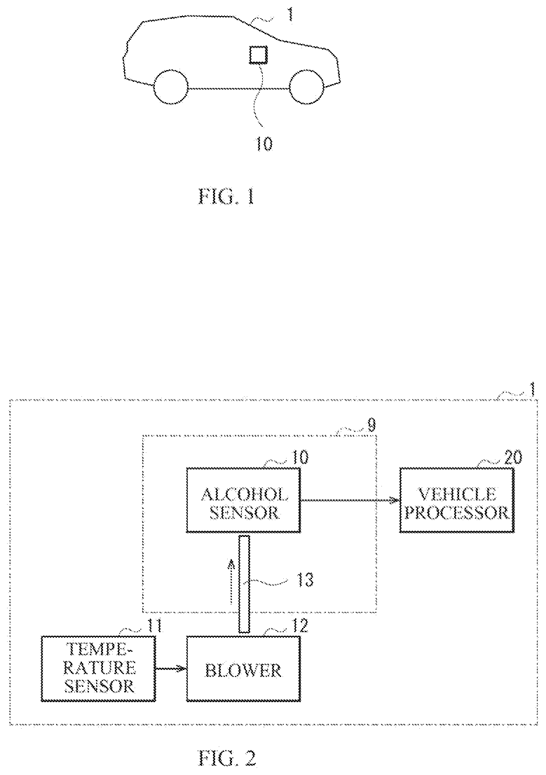

FIG. 2 illustrates a configuration example of example components related to the alcohol sensor 10 in the vehicle 1. The vehicle 1 may include the alcohol sensor 10, a temperature sensor 11, a blower 12, an air duct 13, and a vehicle processor 20. The alcohol sensor 10 and the air duct 13 may be provided inside a later-described space 9 in the vehicle 1.

The temperature sensor 11 may be configured to detect a temperature of a vehicle compartment of the vehicle 1.

In some embodiments, the blower 12 may be an air conditioner of the vehicle 1C. The blower 12 is configured to blow out air whose temperature has been adjusted based on a set temperature set by the occupant and a result of the detection performed by the temperature sensor 11. The blower 12 so adjusts the temperature of the vehicle compartment that the temperature of the vehicle compartment becomes the set temperature by blowing out the air whose temperature has been adjusted.

The air duct 13 may have a tube shape. The air duct 13 is configured to flow a part of the air blown out from the blower 12 toward the alcohol sensor 10.

The vehicle processor 20 may be or may include, for example, an electronic control unit (ECU), and may be configured to control the vehicle 1. Based on the result of the detection performed by the alcohol sensor 10, the vehicle processor 20 may allow the vehicle 1 to travel when an amount of the alcohol component included in the exhaled breath of the driver is equal to or less than a predetermined amount. The vehicle processor 20 may prevent the vehicle 1 from traveling when the amount of the alcohol component included in the exhaled breath of the driver is greater than the predetermined amount. For example, the vehicle processor 20 may not allow a power source such as an engine or a motor to start, when the amount of the alcohol component included in the exhaled breath of the driver is greater than the predetermined amount.

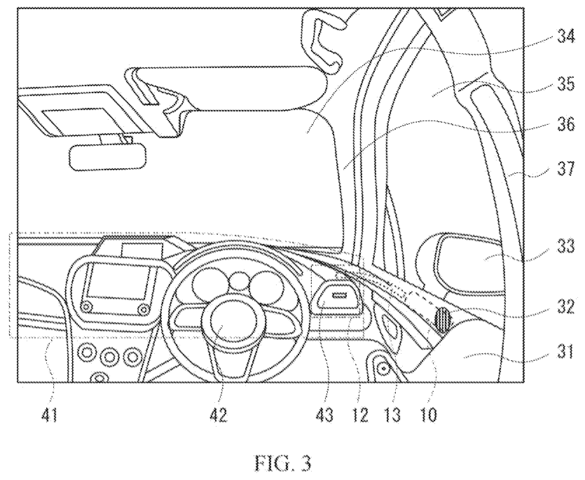

FIG. 3 illustrates a configuration example of the vehicle compartment of the vehicle 1. The vehicle 1 may have a driver's seat on the right side of the vehicle 1. It should be noted that any embodiment of the disclosure is not limited thereto, and the driver's seat may be provided on the left side of the vehicle 1. The vehicle 1 may include a door 31, an inlet 32, a door mirror 33, a windshield 34, a side window 35, a front pillar 36, a center pillar 37, a dashboard 41, a steering wheel 42, and an outlet 43. The door 31 may be provided on a side surface of the vehicle 1, and allow the driver to get on and off the vehicle 1. In the example embodiment, the air duct 13 and the alcohol sensor 10 may be provided inside the door 31. The inlet 32 may be provided at a location, of the door 31, corresponding to the alcohol sensor 10. The inlet 32 may be provided at a location where direct sunlight is less likely to hit or unlikely to hit. The inlet 32 may have a color that, for example, less likely to absorb heat or hardly absorbs heat. For example, the driver seated on the driver's seat may blow his/her breath toward the inlet 32, whereby the alcohol sensor 10 provided inside the door 31 may detect the alcohol component included in the exhaled breath of the driver. The door mirror 33 may be provided on an outer surface of the door 31. The windshield 34 may be provided at the front of the vehicle compartment of the vehicle 1. The side window 35 may be provided in the door 31. The front pillar 36 may support a roof of the vehicle 1 at an end of the windshield 34. The center pillar 37 may support the roof of the vehicle 1 in the vicinity of the middle of the side surface of the vehicle 1. The dashboard 41 may be provided at a lower part of the windshield 34 at the front of the vehicle compartment of the vehicle 1. The steering wheel 42 may be provided at the driver's seat. The outlet 43 may blow out air from the air conditioner of the vehicle 1, and may be provided at a location, of the dashboard 41, close to the door 31.

One end of the air duct 13 may be guided to the blower 12 provided inside the dashboard 41, and the other end of the air duct 13 may be guided to the alcohol sensor 10. The air duct 13 may flow, toward the alcohol sensor 10, a part of the air whose temperature has been adjusted by the blower 12.

In the example embodiment, one end of the air duct 13 may be guided to the blower 12 provided inside the dashboard 41, but any embodiment of the disclosure is not limited thereto. In some embodiments, one end of the air duct 13 may be guided to a location, in the vicinity of the outlet 43, at which the air blown out from the outlet 43 flows. In this case, it is possible for the air duct 13 to cause a part of the air blown out from the outlet 43 to flow to the alcohol sensor 10.

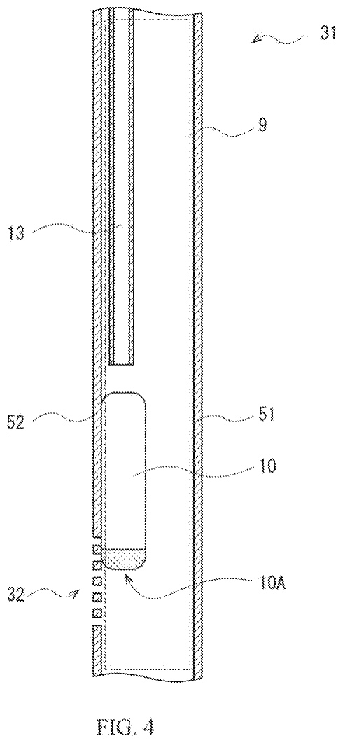

FIG. 4 illustrates an example of a location of the air duct 13 and the alcohol sensor 10 in the door 31. In FIG. 4, a space on the left of the door 31 may be the vehicle compartment of the vehicle 1, and a space on the right of the door 31 may be the outside of the vehicle 1. The door 31 may include an outer panel 51 and an inner panel 52. The outer panel 51 may be an outer surface panel of the door 31 opposed to the outside of the vehicle 1. The inner panel 52 may be an inner surface panel of the door 31 opposed to the vehicle compartment of the vehicle 1. A space 9 may be provided between the outer panel 51 and the inner panel 52. The air duct 13 and the alcohol sensor 10 may be provided in the space 9. An end of the air duct 13 may be guided to the alcohol sensor 10. The alcohol sensor 10 may be in contact with the inner panel 52. The alcohol sensor 10 may be spaced apart from the outer panel 51. The inlet 32 provided on the inner panel 52 may be so opened as to couple the vehicle compartment of the vehicle 1 and the space 9. The alcohol sensor 10 may be so disposed that the inlet 10A of the alcohol sensor 10 is positioned close to the inlet 32 of the inner panel 52.

In one embodiment, the alcohol sensor 10 may serve as an “alcohol sensor”. In one embodiment, the temperature sensor 11 may serve as a “temperature sensor”. In one embodiment, the blower 12 may serve as a “blower”. In one embodiment, the air duct 13 may serve as an “air duct”. In one embodiment, the outer panel 51 may serve as an “outer panel”. In one embodiment, the inner panel 52 may serve as an “inner panel”. In one embodiment, the inlet 32 may serve as an “opening”. In one embodiment, the space 9 may serve as a “space”. In one embodiment, the vehicle processor 20 may serve as a “processor”.

Next, an operation and workings of the vehicle 1 according to the example embodiment will be described.

The example operation of the vehicle 1 will be described with reference to FIG. 2. The temperature sensor 11 may detect the temperature of the vehicle compartment of the vehicle 1. The blower 12 is configured to blow out the air whose temperature has been adjusted based on the set temperature set by the occupant and a result of the detection performed by the temperature sensor 11. The air duct 13 is configured to flow a part of the air blown out from the blower 12 toward the alcohol sensor 10. The alcohol sensor 10 detects the alcohol component included in the exhaled breath of the driver. Based on the result of the detection performed by the alcohol sensor 10, the vehicle processor 20 may allow the vehicle 1 to travel when the amount of the alcohol component included in the exhaled breath of the driver is equal to or less than the predetermined amount, and prevent the vehicle 1 from traveling when the amount of the alcohol component included in the exhaled breath of the driver is greater than the predetermined amount.

As illustrated in FIG. 4, the alcohol sensor 10 may be provided in the space 9 between the outer panel 51 and the inner panel 52. The blower 12 blows out the air whose temperature has been adjusted based on the set temperature set by the occupant and a result of the detection performed by the temperature sensor 11. The air duct 13 flows a part of the air blown out from the blower 12 toward the alcohol sensor 10. Thus, the temperature of the alcohol sensor 10 may be so adjusted as to become a temperature that is within a predetermined temperature range that allows for a high detection accuracy. For example, after the driver of the vehicle 1 sits on the driver's seat, the driver may blow a breath into the inlet 32 provided on the inner panel 52 without directly touching the alcohol sensor 10. Accordingly, the alcohol sensor 10 may detect the alcohol component included in the exhaled breath of the driver. Based on the result of the detection performed by the alcohol sensor 10, the vehicle processor 20 may allow the vehicle 1 to travel when the amount of the alcohol component included in the exhaled breath of the driver is equal to or less than the predetermined amount, and may prevent the vehicle 1 from traveling when the amount of the alcohol component included in the exhaled breath of the driver is greater than the predetermined amount.

The vehicle 1 according to the example embodiment includes the air duct 13 and adjusts the temperature of the alcohol sensor 10, based on the air suppled through the air duct 13 from the blower 12 and whose temperature has been adjusted. This configuration of the vehicle 1 helps to allow the occupant to easily use the alcohol sensor 10. For example, it is difficult to adjust the temperature of the alcohol sensor 10 when the air duct 13 is not provided, which can raise a possibility that the temperature of the alcohol sensor 10 can deviate from the predetermined temperature range that allows for the high detection accuracy in a situation such as a hot summer time or a cold winter time. In addition, for example, when an alcohol sensor carriable by the occupant is to be used and a temperature of the alcohol sensor is low, it is necessary for the occupant to warm the alcohol sensor by, for example, grasping the alcohol sensor with his/her hand, blowing a breath to the alcohol sensor, or bringing the alcohol sensor close to the outlet 43 of the air conditioner, which takes time and effort. In contrast, the vehicle 1 according to the example embodiment includes the air duct 13 and adjusts the temperature of the alcohol sensor 10, based on the air suppled through the air duct 13 from the blower 12 and whose temperature has been adjusted, which helps to allow the temperature of the alcohol sensor 10 to be the temperature that is within the predetermined temperature range that allows for the high detection accuracy. The vehicle 1 utilizes the air supplied from the blower 12 such as the air conditioner, which eliminates the necessity of providing a dedicated temperature adjustment device for the alcohol sensor 10 and achieves a simple configuration accordingly. As described above, the vehicle 1 makes it possible to adjust the temperature of the alcohol sensor 10, which in turn makes it possible to quickly change the temperature of the alcohol sensor 10 to the temperature that is within the predetermined temperature range that allows for the high detection accuracy in a situation such as a hot summer time or a cold winter time. Accordingly, this configuration of the vehicle 1 helps to allow the occupant to easily use the alcohol sensor 10.

In addition, in the vehicle 1 according to the example embodiment, the alcohol sensor 10 may be provided in the space 9 between the outer panel 51 and the inner panel 52. This configuration of the vehicle 1 helps to allow the occupant to easily use the alcohol sensor 10. For example, when an alcohol sensor carriable by the occupant is to be used, there is a possibility that the alcohol sensor can be lost or that the alcohol sensor can be broken, making it difficult for the occupant to use the alcohol sensor. In contrast, in the vehicle 1 according to the example embodiment, the alcohol sensor 10 may be provided in the space 9 between the outer panel 51 and the inner panel 52, preventing the occupant from removing the alcohol sensor 10 and allowing the occupant not to lose the alcohol sensor 10 accordingly. In addition, the alcohol sensor 10 may be protected by the outer panel 51 and the inner panel 52, making it possible to reduce a possibility of being broken by an external force. Further, the alcohol sensor 10 may not be exposed to direct sunlight, reducing a possibility of breakage of the alcohol sensor 10. Accordingly, this configuration of the vehicle 1 helps to allow the occupant to easily use the alcohol sensor 10.

Further, in the vehicle 1 according to the example embodiment, the alcohol sensor 10 may be provided at a location close to the side window 35 in the door 31 of the driver's seat. The location of the alcohol sensor 10 is relatively close to a position of the head of the driver, allowing the driver to easily blows his/her breath toward the inlet 32 of the inner panel 52 of the door 31. Accordingly, this configuration of the vehicle 1 helps to allow the occupant to easily use the alcohol sensor 10.

The vehicle 1 according to the foregoing example embodiment includes the alcohol sensor 10, the blower 12, and the air duct 13. The alcohol sensor 10 is configured to detect the alcohol component included in the exhaled breath of the occupant of the vehicle 1. The blower 12 is configured to blow the air, and adjust the temperature of the air to be blown out from the blower 12. The air duct 13 is configured to flow the air blown out from the blower 12 toward the alcohol sensor 10. Thus, the vehicle 1 makes it possible to quickly change the temperature of the alcohol sensor 10 to the temperature that is within the predetermined temperature range that allows for the high detection accuracy in a situation such as a hot summer time or a cold winter time. Accordingly, this configuration of the vehicle 1 helps to allow the occupant to easily use the alcohol sensor 10.

In some embodiments, the vehicle 1 may further include the temperature sensor 11 configured to perform the detection of the temperature of the vehicle compartment of the vehicle 1. The blower 12 may be configured to adjust the temperature of the air to be blown out from the blower 12, based on a result of the detection performed by the temperature sensor 11. Thus, the vehicle 1 makes it possible to adjust the temperature of the vehicle compartment and the temperature of the alcohol sensor 10 in a situation such as a hot summer time or a cold winter time. Accordingly, this configuration helps to adjust the temperature of the alcohol sensor 10 without making the occupant conscious of adjusting the temperature of the alcohol sensor 10. Accordingly, this configuration of the vehicle 1 helps to allow the occupant to easily use the alcohol sensor 10.

In some embodiments, the vehicle 1 may further include the outer panel 51, the inner panel 52, and the inlet 32. The outer panel 51 may be opposed to the outside of the vehicle 1. The inner panel 52 may be opposed to the vehicle compartment of the vehicle 1. The inlet 32 may be provided on the inner panel 52. The alcohol sensor 10 and the air duct 13 may be provided in the space 9 between the outer panel 51 and the inner panel 52. The inlet 32 may be provided on the inner panel 52 at a location corresponding to the alcohol sensor 10, and couple the vehicle compartment and the space 9. This configuration helps to allow the occupant not to lose the alcohol sensor 10, and to reduce a possibility that the alcohol sensor 10 is broken. Accordingly, this configuration of the vehicle 1 helps to allow the occupant to easily use the alcohol sensor 10.

In some embodiments, the vehicle 1 may further include the vehicle processor 20 configured to control the vehicle 1. The vehicle processor 20 may allow the vehicle 1 to travel when the amount of the alcohol component is equal to or less than the predetermined amount, and prevent the vehicle 1 from traveling when the amount of the alcohol component is greater than the predetermined amount. Accordingly, this configuration helps to reduce a possibility that the driver performs drunk driving.

The vehicle according to the example embodiment includes: the alcohol sensor configured to detect the alcohol component included in the exhaled breath of the occupant of the vehicle; the blower configured to blow the air, and adjust the temperature of the air to be blown out from the blower; and the air duct configured to flow the air blown out from the blower toward the alcohol sensor. This configuration helps to allow the occupant to easily use the alcohol sensor.

In some embodiments, the vehicle may further include the temperature sensor configured to perform the detection of the temperature of the vehicle compartment of the vehicle, and the blower may be configured to adjust the temperature of the air to be blown out from the blower, based on a result of the detection performed by the temperature sensor. This configuration helps to allow the occupant to easily use the alcohol sensor.

In some embodiments, the vehicle may further include: the outer panel opposed to the outside of the vehicle; the inner panel opposed to the vehicle compartment of the vehicle; and the inlet provided on the inner panel. The alcohol sensor and the air duct may be provided in the space between the outer panel and the inner panel. The inlet may be provided on the inner panel at a location corresponding to the alcohol sensor, and couple the vehicle compartment and the space. This configuration helps to allow the occupant to easily use the alcohol sensor.

In some embodiments, the vehicle may further include the vehicle processor configured to control the vehicle, and allow the vehicle to travel when the amount of the alcohol component is equal to or less than the predetermined amount, and prevent the vehicle from traveling when the amount of the alcohol component is greater than the predetermined amount. This configuration helps to reduce a possibility that the driver performs drunk driving.

In the above-described example embodiment, the air duct 13 and the alcohol sensor 10 may be provided inside the door 31, but any embodiment of the disclosure is not limited thereto. In some embodiments, the air duct 13 and the alcohol sensor 10 may be provided inside the front pillar 36 as illustrated in FIG. 5. In the present modification example, one end of the air duct 13 may be guided to the blower 12 provided inside the dashboard 41, and the other end of the air duct 13 may be guided to the alcohol sensor 10. The air duct 13 may flow a part of the air whose temperature has been adjusted by the blower 12 toward the alcohol sensor 10. The vehicle 1 according to the present modification example may include an inlet 32A. The inlet 32A may be provided at a location, of the front pillar 36, corresponding to the alcohol sensor 10. In the present modification example, the inlet 32A may be provided at a location in the vicinity of the middle of the front pillar 36 in a longitudinal direction. The inlet 32A may be provided at a location where the direct sunlight is less likely to hit or unlikely to hit. The inlet 32A may have a color that, for example, less likely to absorb heat or hardly absorbs heat. For example, the driver seated on the driver's seat may blow his/her breath toward the inlet 32A, whereby the alcohol sensor 10 provided inside the front pillar 36 may detect the alcohol component included in the exhaled breath of the driver. In this modification example, the air duct 13 and the alcohol sensor 10 may be provided inside the front pillar 36, but any embodiment of the disclosure is not limited thereto. In some embodiments, the air duct 13 and the alcohol sensor 10 may be provided inside the center pillar 37. In this case, one end of the air duct 13 may be guided to, for example, an unillustrated blower provided for a rear seat. The air duct 13 may flow a part of the air whose temperature has been adjusted by the blower for the rear seat toward the alcohol sensor 10.

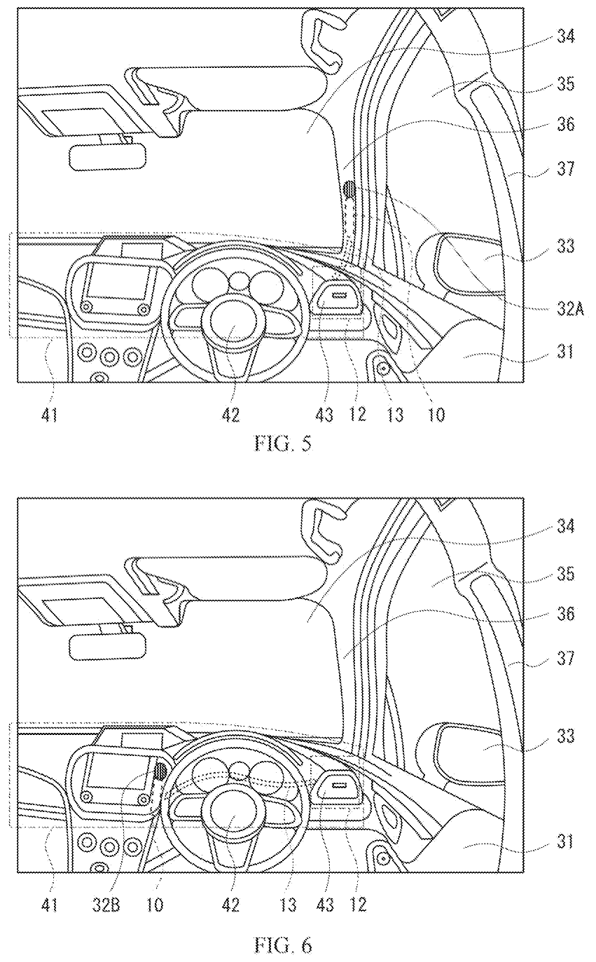

In some embodiments, the air duct 13 and the alcohol sensor 10 may be provided inside the dashboard 41 as illustrated in FIG. 6. In the present modification example, one end of the air duct 13 may be guided to the blower 12 provided inside the dashboard 41, and the other end of the air duct 13 may be guided to the alcohol sensor 10. The vehicle 1 according to the present modification example may include an inlet 32B. The inlet 32B may be provided at a location, of the dashboard 41, corresponding to the alcohol sensor 10. In the present modification example, the inlet 32B may be provided at a location, of the dashboard 41, close to the steering wheel 42. The inlet 32B may be provided at a location where the direct sunlight is less likely to hit or unlikely to hit. The inlet 32B may have a color that, for example, less likely to absorb heat or hardly absorbs heat. For example, the driver seated on the driver's seat may blow his/her breath toward the inlet 32B, whereby the alcohol sensor 10 provided inside the dashboard 41 may detect the alcohol component included in the exhaled breath of the driver.

In the above-described example embodiment, the air conditioner of the vehicle 1 may be used as the blower 12, but any embodiment of the disclosure is not limited thereto. In some embodiments, a defroster may be used as the blower. In the following, a vehicle 1C according to the present modification example will be described in detail.

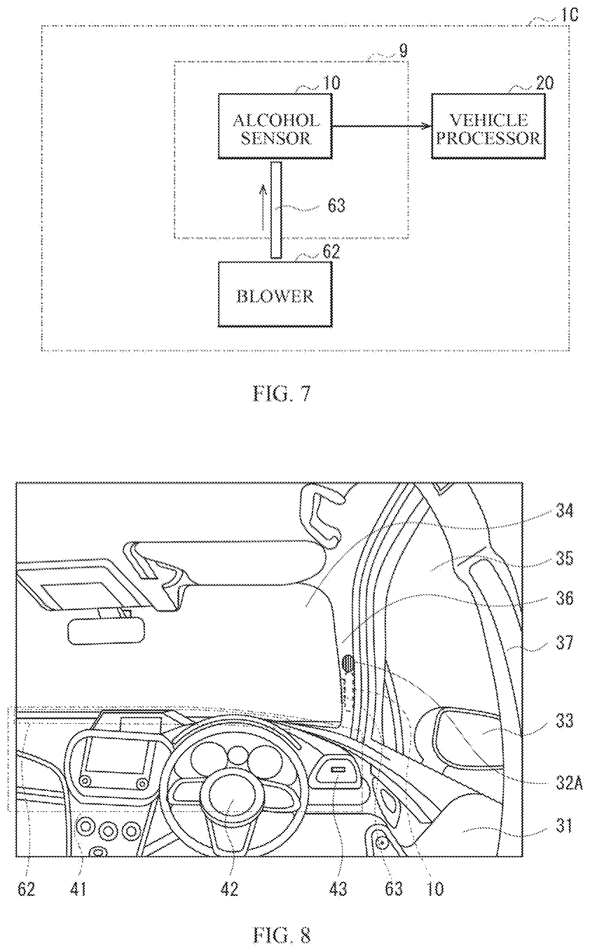

FIG. 7 illustrates a configuration example of example components related to the alcohol sensor 10 in the vehicle 1C. FIG. 8 illustrates a configuration example of a vehicle compartment of the vehicle 1C. The vehicle 1C may include a blower 62 and an air duct 63. In the present modification example, although it is not limited thereto, the air duct 63 and the alcohol sensor 10 may be provided inside the front pillar 36.

The blower 62 may be a defroster that reduces or eliminates fogging on an inner side of the windshield 34. The blower 62 may be configured to blow, for example, warm air with less water vapor toward the windshield 34.

The air duct 63 may be configured to flow, toward the alcohol sensor 10, a part of the air blown out from the blower 62 and flowing along the inner side of the windshield 34. The air duct 63 may be an opening provided on the front pillar 36. The air duct 63 may couple the space, inside the front pillar 36, in which the alcohol sensor 10 is provided and the vehicle compartment.

The blower 62 may blow, for example, warm air toward the windshield 34. The air blown out from the blower 62 may flow along the inner side of the windshield 34, and a part of the air flowing along the inner side of the windshield 34 may flow toward the alcohol sensor 10 through the air duct 63. This configuration thus helps to adjust the temperature of the alcohol sensor 10.

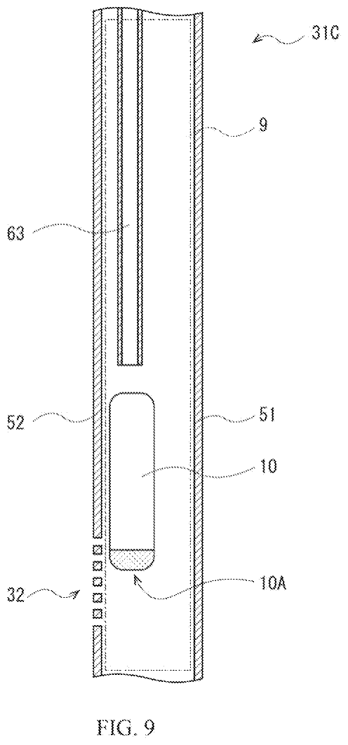

In the above-described example embodiment, the alcohol sensor 10 may be so disposed as to be in contact with the inner panel 52, but any embodiment of the disclosure is not limited thereto. In some embodiments, the alcohol sensor 10 may be so disposed as to be spaced apart from the inner panel 52 as illustrated in FIG. 9.

The techniques according to the example embodiment and its modification examples described above may be combined in any combination unless any contradiction occurs.

Although some example embodiments of the disclosure have been described in the foregoing by way of example with reference to the accompanying drawings, the disclosure is by no means limited to the embodiments described above. It should be appreciated that modifications and alterations may be made by persons skilled in the art without departing from the scope as defined by the appended claims. The disclosure is intended to include such modifications and alterations in so far as they fall within the scope of the appended claims or the equivalents thereof.

For example, in the above example embodiment and its modification examples, the technology may be applied to the vehicle having a driver's seat on the right side, but any embodiment of the disclosure is not limited thereto. In some embodiments, the technology may be applied to a vehicle having a driver's seat on the left side.

The example effects described herein are merely examples, and effects of the disclosure are not limited to the effects described herein. Accordingly, any other effect may be achieved by any embodiment of the disclosure.

It is possible to achieve at least the following configurations from the foregoing example embodiment and its modification examples of the disclosure.

(1)

A vehicle including:
an alcohol sensor configured to detect an alcohol component included in exhaled breath of an occupant of the vehicle;
a blower configured to blow air, and adjust a temperature of the air to be blown out from the blower; and
an air duct configured to flow the air blown out from the blower toward the alcohol sensor.
   (2)

The vehicle according to (1), further including a temperature sensor configured to perform a detection of a temperature of a vehicle compartment of the vehicle, in which
the blower is configured to adjust the temperature of the air to be blown out from the blower, based on a result of the detection performed by the temperature sensor.
   (3)

The vehicle according to (1) or (2), further including:
an outer panel opposed to outside of the vehicle; and
an inner panel opposed to a vehicle compartment of the vehicle and having an opening, in which
the alcohol sensor and the air duct are provided in a space between the outer panel and the inner panel, and
the opening is provided on the inner panel at a location corresponding to the alcohol sensor, and couples the vehicle compartment and the space.
   (4)

The vehicle according to (3), in which
the outer panel includes a panel of an outer surface of a door of the vehicle, and
the inner panel includes a panel of an inner surface of the door of the vehicle.
   (5)

The vehicle according to any one of (1) to (4), further including a processor configured to control the vehicle, and allow the vehicle to travel when an amount of the alcohol component is equal to or less than a predetermined amount, and prevent the vehicle from traveling when the amount of the alcohol component is greater than the predetermined amount.

The vehicle processor 20 illustrated in FIGS. 2 and 7 is implementable by circuitry including at least one semiconductor integrated circuit such as at least one processor (e.g., a central processing unit (CPU)), at least one application specific integrated circuit (ASIC), and/or at least one field programmable gate array (FPGA). At least one processor is configurable, by reading instructions from at least one machine readable non-transitory tangible medium, to perform all or a part of functions of the vehicle processor 20. Such a medium may take many forms, including, but not limited to, any type of magnetic medium such as a hard disk, any type of optical medium such as a CD and a DVD, any type of semiconductor memory (i.e., semiconductor circuit) such as a volatile memory and a non-volatile memory. The volatile memory may include a DRAM and a SRAM, and the nonvolatile memory may include a ROM and a NVRAM. The ASIC is an integrated circuit (IC) customized to perform, and the FPGA is an integrated circuit designed to be configured after manufacturing in order to perform, all or a part of the functions of the vehicle processor 20 illustrated in FIGS. 2 and 7.

1. A vehicle comprising:
an alcohol sensor configured to detect an alcohol component included in exhaled breath of an occupant of the vehicle;
a blower configured to blow air, and adjust a temperature of the air to be blown out from the blower; and
an air duct configured to flow the air blown out from the blower toward the alcohol sensor.

2. The vehicle according to claim 1, further comprising a temperature sensor configured to perform a detection of a temperature of a vehicle compartment of the vehicle, wherein
the blower is configured to adjust the temperature of the air to be blown out from the blower, based on a result of the detection performed by the temperature sensor.

3. The vehicle according to claim 1, further comprising:
an outer panel opposed to outside of the vehicle; and
an inner panel opposed to a vehicle compartment of the vehicle and having an opening, wherein
the alcohol sensor and the air duct are provided in a space between the outer panel and the inner panel, and
the opening is provided on the inner panel at a location corresponding to the alcohol sensor, and couples the vehicle compartment and the space.

4. The vehicle according to claim 3, wherein
the outer panel comprises a panel of an outer surface of a door of the vehicle, and
the inner panel comprises a panel of an inner surface of the door of the vehicle.

5. The vehicle according to claim 1, further comprising a processor configured to control the vehicle, the processor being configured to allow the vehicle to travel when an amount of the alcohol component is equal to or less than a predetermined amount, and prevent the vehicle from traveling when the amount of the alcohol component is greater than the predetermined amount.
